(19) United States
(12) Patent Application Publication
(10) Pub. No.: US 2010/0305557 A1
(43) Pub. Date: Dec. 2, 2010

(54) HTA Sheath with Removable Scope

(76) Inventor: Michael S. H. Chu, Brookline, MA (US)

Correspondence Address: FAY KAPLUN & MARCIN, LLP, 150 BROADWAY, SUITE 702, NEW YORK, NY, 10038, US

(21) Appl. No.: 12/729,872

(22) Filed: Mar. 23, 2010

Related U.S. Application Data
(60) Provisional application No. 61/183,108, filed on Jun. 2, 2009.

(51) Int. Cl. A61B 18/04

(52) U.S. Cl. 606/27

(57) ABSTRACT

An ablation device comprises an elongated introducer sized for insertion into a hollow organ via a natural opening, the introducer extending to a distal end which, when in an operative position, is within the hollow organ, the introducer including and a delivery lumen extending from a proximal end connectable to a source of ablation fluid to a distal delivery opening at the distal end in combination with a working channel extending to a working channel distal opening at the distal end, the working channel being sized and shaped so that, when a flexible instrument to be inserted into the hollow organ is received therein, a space between an outer surface of the flexible instrument and an inner wall of the working channel forms a return lumen extending from the working channel distal opening to a proximal return port and a fluid supply unit coupled to the introducer supplying ablation fluid to the delivery lumen.

### PRIORITY CLAIM

The present application claims the priority to the U.S. Provisional Application Ser. No. 61/183,108, entitled “HTA Sheath with Removable Scope” filed on Jun. 2, 2009. The specification of the above-identified application is incorporated herewith by reference.

### BACKGROUND

terine bleeding is often treated through ablation of the endometrium. In hydro-thermal ablation (“HTA”), heated fluid (e.g., saline solution) is supplied to the uterus via a device inserted through the cervical canal to ablate the endometrium. During the procedure fluid is withdrawn from the uterus to permit the introduction of fresh or reheated ablation fluid. However, debris such as coagulated tissue can clog the removal lumen hindering desired fluid circulation.

### SUMMARY OF THE INVENTION

The present invention is directed to an ablation device comprising an elongated introducer sized for insertion into a hollow organ via a natural opening, the introducer extending to a distal end which, when in an operative position, is within the hollow organ, the introducer including and a delivery lumen extending from a proximal end connectable to a source of ablation fluid to a distal delivery opening at the distal end in combination with a working channel extending to a working channel distal opening at the distal end, the working channel being sized and shaped so that, when a flexible instrument to be inserted into the hollow organ is received therein, a space between an outer surface of the flexible instrument and an inner wall of the working channel forms a return lumen extending from the working channel distal opening to a proximal return port and a fluid supply unit coupled to the introducer supplying ablation fluid to the delivery lumen.

### BRIEF DESCRIPTION OF DRAWINGS

FIG. 1 shows a partial cross-sectional view of an ablation system according to an embodiment of the invention;

FIG. 2 shows an enlarged partial cross-sectional view of a proximal end of the device of FIG. 1 in a first configuration;

FIG. 3 shows an enlarged partial cross-sectional view of a distal end of the device of FIG. 1 in a first configuration;

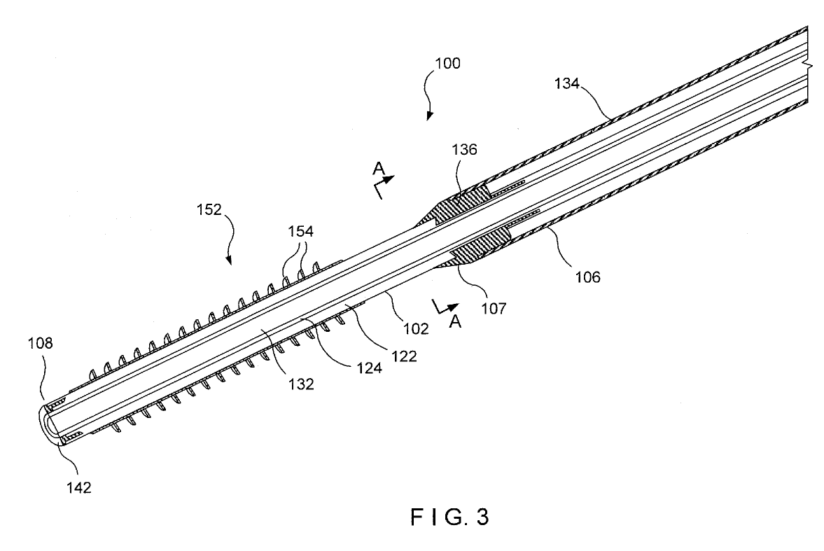

FIG. 4 shows a cross sectional view taken along line A-A of FIG. 3; and

FIG. 5 shows a partial cross-sectional view of FIG. 2 in a second configuration.

### DETAILED DESCRIPTION

The present invention may be further understood with reference to the following description and the related appended drawings, wherein like elements are provided with the same reference numerals. The present invention describes a system and method for selectively inserting an endoscope or other device known in the art into a hydro-thermal ablation (“HTA”) device to prevent a clogging of a withdrawal lumen thereof. The present invention relates to a system and method for thermally ablating an inner surface of a hollow organ. In particular, the present invention relates to devices for ablating the endometrial lining of the uterus. However, those of skill in the art will understand that the present invention, or components thereof, may be utilized in prostate treatment (microwave or cyroablation) systems, irrigation systems or other procedures which require infusion of fluid into a internal body structures. As used in this application the terms proximal and distal refer to a direction toward and away from a user of the device, respectively. Thus, the proximal end of the device is an end which remains outside the body accessible to a user while the distal end is inserted to a target location within the body.

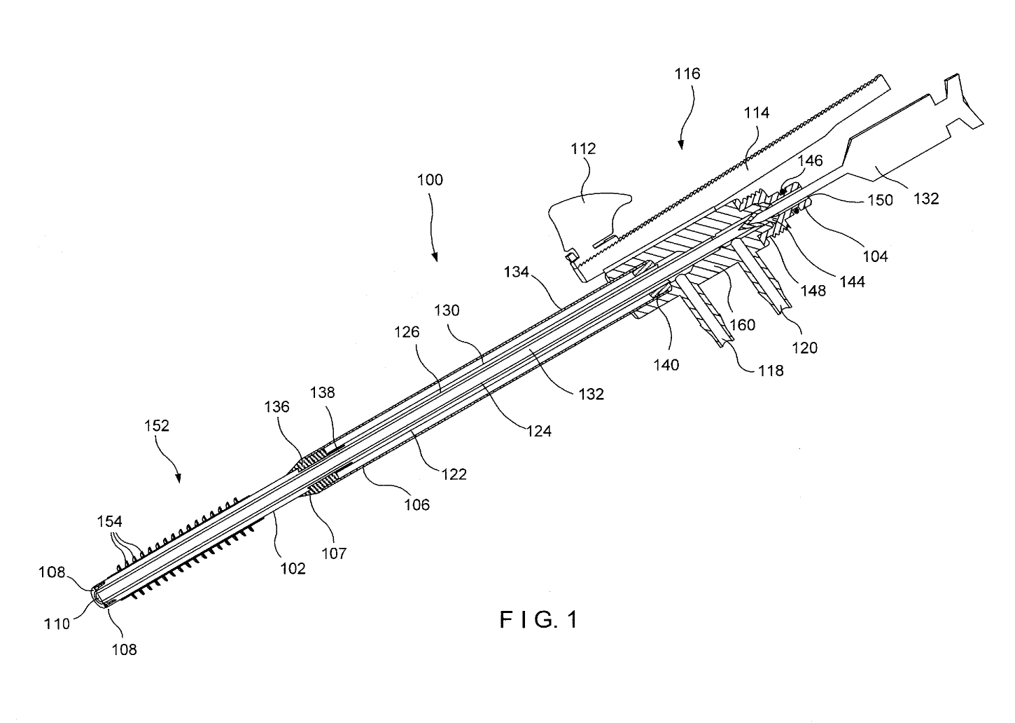

FIG. 1 depicts an introducer 100 for an ablation system according to an exemplary embodiment of the invention comprising an elongated body 106 extending from a proximal end 104 which remains outside the body at all times to a distal end 108 which, when in an operative position, opens into a uterus of a living body. A tapered portion 107 defines a transition between an increased diameter elongated body 106 and a decreased diameter sheath 102 forming a distal portion of the introducer 100. The tapered portion 107 comprises a connector 136 connecting the elongated body 106 to the sheath 102 to make gradual the transition between the larger diameter elongated body 106 which is formed as a sheath 134 and the sheath 102 which has a smaller outside diameter. The connector 136 may be permanently bonded in place to provide a secure attachment as would be understood by those skilled in the art. In use, the introducer 100 is coupled to a known thermal ablation system including a supply of heated fluid and a system for withdrawing the fluid from the body. For example, the introducer 100 of the present invention may be employed with a thermal ablation system as described in U.S. Patent Application Ser. No. 60/987,913, filed Nov. 14, 2007, entitled “Thermal Ablation System,” and naming as inventors Robert Bouthillier, Michael P. Fusaro, Joseph M. Gordon, Stephen S. Keaney, Brian Maclean, Andrew W. Marsella, David Robson and Boris Shapeton (“the '913 application). The entire disclosure of the '913 application is hereby incorporated by reference herein. Generally, a the thermal ablation system may include a console receiving ablation fluid from a source such as an intravenous bag (“IV bag”) (not shown) hung at a height selected to provide a desired pressure. As described above, known thermal ablation system permits one or both of recirculating fluids received from the return lumen back to the delivery lumen and forwarding fluids from the return lumen to a waste collection reservoir for permanent removal from the system. The console comprises a housing encasing electronic circuitry and providing a user interface for displaying content (e.g., instructions, procedural data, warnings, etc.) and receiving user input and the user interface may comprise a display screen (e.g., LCD) and a keypad for submitting input to the console, etc. The console effectively controls the flow of fluid into and out of the introducer 100.

The introducer 100 is coupled to the thermal ablation system (not shown) via a delivery inlet 118 and a return outlet 120 to deliver fluid to the uterus and to return the fluid from the uterus, respectively. The delivery inlet 118 and the return outlet 120 are respectively coupled to a delivery lumen 122 and a return lumen 124 both extending longitudinally through the introducer 100. A proximal end of the return lumen 124 is fluidly connected to the return outlet 120 while remaining open to a valve 144, as will be described in greater detail below. A proximal end of the delivery lumen 122 is fluidly connected to the delivery inlet 118 and remains fluidly sealed from the valve 144. The delivery lumen 122 and the return lumen 124 extend to openings at the distal end 108 of the sheath which open into the uterus when the introducer is in an operative position.

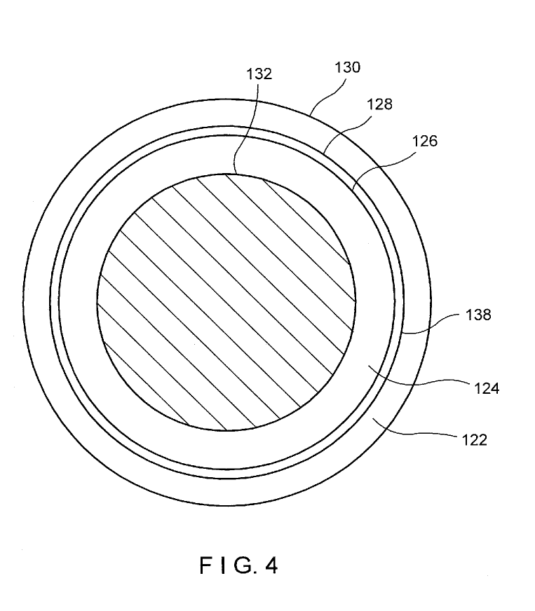

As shown in greater detail in the cross-sectional view of FIG. 4, a return lumen 124 is formed as an annular space surrounding an endoscope or other instrument 132 inserted through a working channel formed within a first wall 126. The working channel formed by the first wall 126 extends proximally past the return outlet 120 to a proximal end in which a valve 144 is mounted. In this manner, as will be described in greater detail below, fluid can be drawn out of the return lumen 124 via the return outlet 120 while an endoscope 132 or other device known in the art may be inserted into the return lumen 124 via the valve 144. The first wall 126 is preferably formed as a unitary element formed of a metal such as stainless steel or other suitable material. In an exemplary embodiment, the first wall 126 is formed so that an inner diameter of the working cannel is approximately 4.394 mm. to accommodate an endoscope 132 having a maximum outer diameter of approximately 3.20 mm. When an endoscope 132 of this size is inserted into the working channel, a return lumen 124 having a cross-sectional area of approximately 7.10 mm² remains available for the removal of fluids from the uterus. Specifically, a desired thickness of the return lumen 124 is approximately 0.61 mm or greater. Those skilled in the art will understand that if a diameter of the endoscope 132 is smaller than 3.20 mm., the area remaining for the return lumen 124 will be increased permitting larger pieces of debris to pass therethrough.

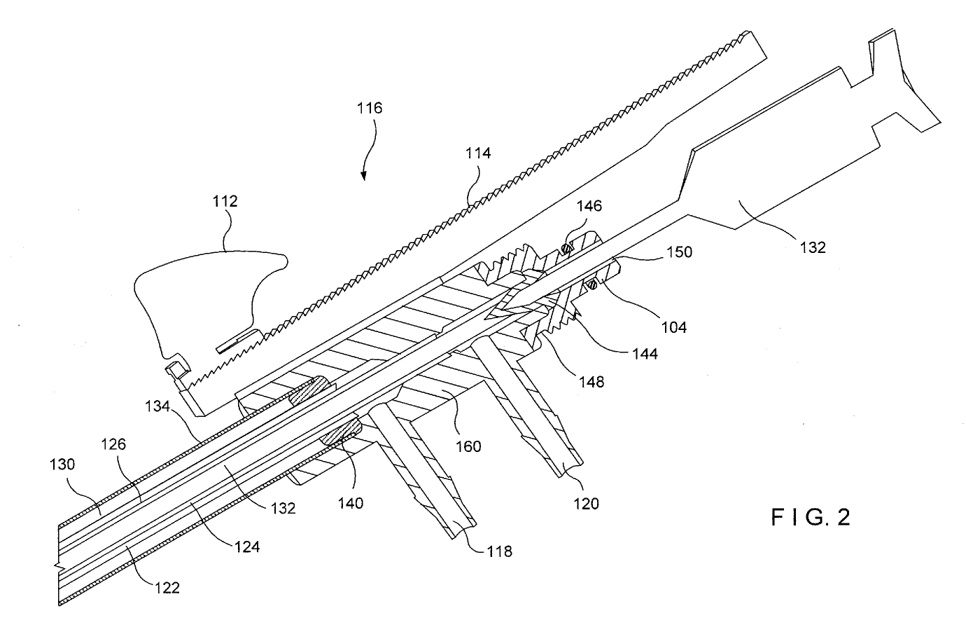

A second wall 128 formed concentrically around the first wall 126 is spaced from the first wall by a predetermined distance defining an air gap 138 therebetween. In one exemplary embodiment, an inner diameter of the second wall 128 is approximately 4.5 to 4.6 mm and may, more specifically, be approximately 5.572 mm. The second wall 128 is preferably formed as a unitary element formed of a polymer such as polycarbonate or other suitable material. Those skilled in the art will understand that the air gap 138 provides insulation to minimize heat transfer to non-targeted tissue to prevent burns, etc. The second wall 128 and the air gap 138 extend from a proximal end comprising a plug 140 to a distal end comprising a tip 142, being sealed at both ends thereof to prevent any foreign matter from entering thereinto and to secure the air within the air gap 138. As shown in the embodiment of FIGS. 1, 2 and 5, the first wall 126 extends proximally of the plug 140 to the proximal end 104 of the introducer 100. A third wall 130 is formed concentrically around a distal length of the introducer 100, extending from a proximal end opening into the delivery outlet 118 to the distal end 108 open to the body in an operative configuration. The third wall 130 defines an outer wall of the air gap 138 extending therethrough. In an exemplary embodiment, the third wall 130 is formed of a polymer such as, for example, polycarbonate. The second wall 128 defines an outer wall of the return lumen 124 extending therewithin. More specifically, an outer wall of the delivery lumen 122 is defined by the second wall 128 and an inner wall of the delivery lumen 122 is defined by the first wall 126. In a preferred embodiment, a diameter of the third wall 130 is approximately 5.31 mm. and the thickness of the delivery lumen is therefore approximately 0.381 mm.

The introducer 100 comprises a handle 160 located on a proximal portion thereof, the handle 160 further comprising a scope connector 148 for receiving a visualization device, such as the endoscope 132. The handle 160 facilitates holding and manipulation of the introducer 100 with a single hand while the operator uses his free hand to interface with a console (not shown) controlling the visualization device, manipulate the patient's anatomy, etc. The scope connector 148 according to this embodiment is disposed on a proximal end of the introducer 100 and provides an attachment point for the endoscope 132 via an adaptor (not shown) so that the endoscope 132 may pass distally through the return lumen 124 to reside within or extend distally from the distal end 108. Thus, the operator may visually monitor insertion of the introducer 100 into the uterus.

The exemplary embodiment of the present invention provides that the return lumen 124 comprises a thickness greater than the delivery lumen 122 in order to accommodate debris and other material flowing therethrough. In this manner, the return lumen 124 permits an unobstructed flow therethrough without clogging due to debris being caught therein. The present invention further allows for an embodiment wherein the endoscope 132 is completely removed from the introducer 100 in order to provide a completely unobstructed return lumen 124. The scope connector 148 secures the endoscope 132 to an end cap 150 via a screw thread 149. Specifically, the screw thread 149 formed along an outer surface of the scope connector is sized and shaped to engage threading formed on a wall of a lumen passing through the scope adaptor (not shown). Thus, the scope adaptor can be screwed into engagement with the scope connector 148. A proximal portion (not shown) of the adaptor is provided with a connector specific to a type of endoscope being passed therethrough. Thus, the scope adaptor (not shown) permits the engagement of various types of endoscopes with the threaded scope connector 148, as those skilled in the art will understand. Accordingly, in order to remove the endoscope from engagement with the introducer, the scope adaptor (not shown) may be unscrewed from the scope connector 148.

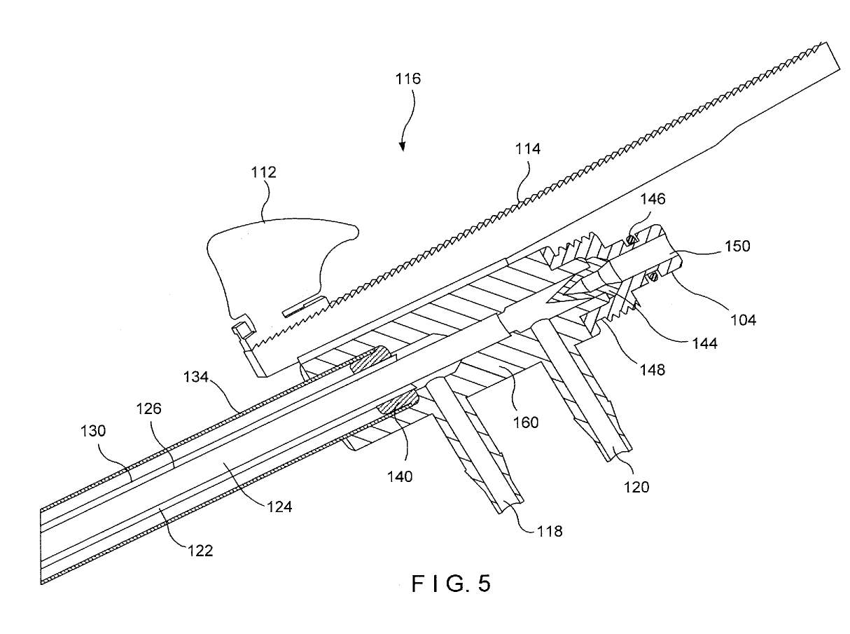

The screw thread 149 of the scope connector 148, when employed with an O-ring 146, as described in greater detail hereinafter, serves as a secondary seal for the scope adaptor (not shown). Specifically, the scope connector 148 comprises a valve 144 formed as a duck-bill type valve, as those skilled in the art will understand. The valve 144 is a self-sealing valve that allows the endoscope 132 to be selectively received therethrough. When the endoscope 132 is removed from the return lumen 124, as shown in FIG. 5, the valve 144 assumes a fluidly sealed configuration where no foreign matter is permitted therepast. The valve 144 further functions as a safety device, preventing a physician or other user of the introducer 100 from being exposed to the heated saline and biological matter. The valve 144 is formed of medical grade silicone in order to be able to withstand the temperature of the heated saline. Because the exemplary embodiment of the present invention permits a range of endoscopes to be employed therewith, the valve 144 is also sized to permit endoscopes ranging from approximately 2.54-3.30 mm. in diameter therethrough. The valve 144 is further provided with the O-ring 146 to provide a secondary means for sealingly securing the scope adaptor (not shown) and the endoscope 132 to the introducer 100 in an operative configuration. The O-ring 146 is formed as a projection on an outer surface of the scope connector 148. Specifically, the O-ring 146 is formed as a loop of elastomer with an o-shaped cross-section to form a mechanical seal around the valve 144 and the endoscope 132 inserted therein. In an alternate embodiment, the valve 144 may also be formed as a touhy-borst valve, wherein an additional step of tightening of a grommet (not shown) employed therewith must be performed, as those skilled in the art will understand.

A distal length of the introducer 100 is provided with a seal 152 comprising a plurality of circumferential ribs 154 located at equidistant intervals thereover. It is noted however, that any spacing interval may be provided between adjacent ribs 154. Furthermore, the ribs may be preformed directly over the sheath 102 or, alternatively, may be formed on a base (not shown) selectively placed and bonded over a distal length of the sheath. The ribs 154 may abut radially out of the sheath 102 by a length sufficient to engage walls of a cervical canal to prevent fluid leakage from the uterus, as those skilled in the art will understand. Furthermore, any plurality of ribs 154 may be employed without deviating from the spirit and scope of the present invention. The ribs 154 may be formed of medical grade silicone. further be permitted to deflect proximally or distally with respect to a longitudinal axis extending through the sheath 102. In this manner, the seal 152 may accommodate the dimension of and frictionally engage the cervical canal without causing trauma thereto. Furthermore, the deflecting capability of the ribs 154 permits the seal 152 to dislodge any tissue or debris caught in the cervical canal. The brushing action may be accomplished by inserting and subsequent manual manipulation of the sheath 102 during a hysteroscopy.

The introducer 100 further comprises a tenaculum stabilizer 116 on the handle 160. During the ablation procedure, a tenaculum (not shown) may be placed around the cervical canal and coupled to the introducer 100 to ensure that the introducer 100 remains at a desired position and depth within the uterus and is not inadvertently withdrawn therefrom. That is, the tenaculum stabilizer 116 ensures that the distal end 108 of the sheath 102 is not withdrawn proximally from the uterus during the procedure to prevent non-targeted tissue from being exposed to the ablation fluid. When the tenaculum is coupled to the tenaculum stabilizer 116, the operator selects a tension to be applied between the tenaculum and the introducer 100 by moving a fin 112 along a rail 114. A positioning mechanism (e.g., ratchet, latch, clip, etc.) may be used to maintain a position of the fin 112 relative to the rail 114, as would be understood by those skilled in the art. Such a tenaculum stabilizer device is also described in U.S. Patent Application Ser. No. 60/987,913 as indicated previously and incorporated herein by reference.

In use, the introducer 100 is moved to a desired position with a distal end 108 thereof within the uterus and the seal 152 seated within a distal portion of the cervical canal (not shown). Specifically, only a distal portion comprising the sheath 102 is received within the cervical canal. Those skilled in the art will understand that the increased diameter of the sheath 134 of the elongated body 106 may cause trauma to the cervical canal. At any point of the procedure, the endoscope 132 may be inserted into the valve 144 and through the return lumen 124 to provide visualization of the target location to a user of the introducer 100. The delivery inlet 118 and the return outlet 120 may then be connected to a source of heated ablation fluid and a reservoir, respectively. The heated ablation fluid may then be delivered to the uterus via the delivery lumen 122. Since the delivery lumen 122 is located radially distally of the return lumen 124, fluid flowing thereoutof does not obstruct the return lumen 124. Once the ablation fluid has been left in the uterus for a predetermined period of time, a suction is applied to the return lumen 124 to cause fluid and ablated tissue to be drawn thereinto. The delivery lumen 122 and the return lumen 124 may be actuated simultaneously to permit a constant flow of heated ablation fluid through the uterus. Upon entry into the return lumen 124, the heated saline and any ablated tissue may travel along a path defined around a periphery of the endoscope 132 to the return outlet 120. Alternatively, the endoscope 132 may be removed at this stage to permit a greater opening for heated saline and ablated tissue to flow thereoutof. Still further, the endoscope 132 may be removed only upon detection of an obstruction in the return lumen 124, wherein the obstruction may be indicated to a user of the device via a signaling means known in the art. The exemplary embodiment of the present invention further permits that, upon clearing of the obstruction, the endoscope 132 may be reinserted into the return lumen 124. Still further, the endoscope 132 may be moved proximally but not completely withdrawn from the lumen 132 to clear any obstructions near the return outlet 120. It is also envisioned that the endoscope 132 not be employed at all at any stage of the ablation procedure to permit a maximized return lumen 124 at all times.

There are many modifications of the present invention which will be apparent to those skilled in the art without departing from the teaching of the present invention. For example, although the delivery lumen 122 and the return lumen 124 are shown with substantially circular walls, it is submitted that any suitable shape may be employed without deviating from the spirit and scope of the present invention such as, for example, D-shaped lumens, elliptical lumens, etc. The introducer 100 may optionally include a vision system to allow visualization of the operative area. Those of skill in the art will understand that the vision system may be substantially similar to the systems in conventional endoscopes (e.g., fiber optic or CCD-based systems). Alternatively, users may rely on the vision system of an endoscope or other instrument inserted through the introducer 100, as those skilled in the art will understand. It is therefore noted that the embodiments shown are shown for illustrative and descriptive purposes only and are not intended to describe the bounds of the present invention which is to be limited only by the scope of the claims appended hereto.

What is claimed is:

1. An ablation device, comprising:
an elongated introducer sized for insertion into a hollow organ via a natural opening, the introducer extending to a distal end which, when in an operative position, is within the hollow organ, the introducer including:
a delivery lumen extending from a proximal end connectable to a source of ablation fluid to a distal delivery opening at the distal end; and
a working channel extending to a working channel distal opening at the distal end, the working channel being sized and shaped so that, when a flexible instrument to be inserted into the hollow organ is received therein, a space between an outer surface of the flexible instrument and an inner wall of the working channel forms a return lumen extending from the working channel distal opening to a proximal return port; and
a fluid supply unit coupled to the introducer supplying ablation fluid to the delivery lumen.

2. The thermal ablation device of claim 1, further comprising:
a seal extending around a portion of the introducer which, when the introducer is in the operative position, is seated within the opening of the hollow organ so that the seal engages the tissue of the opening around a circumference thereof, the seal including a plurality of flexible members separated from one another along a length of the elongated introducer and projecting radially outward from an outer surface of the introducer.

3. The thermal ablation device of claim 2, wherein the flexible members are substantially disc-shaped and are deflectable proximally and distally when compressed by contact with surrounding tissue.

4. The thermal ablation device of claim 1, further comprising:
an air space extending concentrically around the return lumen.

5. The thermal ablation device of claim 1, wherein the hollow organ is a uterus.

6. The thermal ablation device of claim 1, further comprising:
a handle coupled to a proximal end of the introducer including a grip and an instrument connector extending proximally therefrom for connecting to an instrument inserted therethrough into the working channel.

7. The thermal ablation device of claim 6, wherein the scope connector further comprises a valve sealing a distal end of the working channel.

8. The thermal ablation device of claim 7, wherein the valve is one of a duck-bill type valve and a touhy-borst valve.

9. The thermal ablation device of claim 9, wherein, in an operative configuration with an instrument inserted therethrough, the valve fluidly seals around a periphery of the instrument.

10. The thermal ablation device of claim 6, further comprising:
a tenaculum stabilizer on a proximal portion of the introducer.

11. The thermal ablation device of claim 1, wherein the device is configured for thermal ablation of the uterus, further comprising a tapered connector connecting a distal portion of the introducer to a proximal portion thereof, the tapered connector being located so that, when the introducer is in an operative position with the distal end thereof in the uterus, the tapered connector abuts a proximal end of the cervix.

12. A method, comprising:
inserting an ablation device to an operative position in which in which a distal end of an introducer of the device is positioned within a patient's uterus, the introducer including a delivery lumen extending to a distal delivery opening at the distal end and a working channel extending to a working channel distal opening at the distal end;
inserting a flexible instrument into the working channel and advancing the instrument through the working channel to the working channel distal opening;
supplying ablation fluid to the uterus via the distal delivery opening of the delivery lumen; and
withdrawing fluid from the uterus via a return lumen formed as an annular space between an inner wall of the working channel and an outer surface of the flexible instrument.

13. The method of claim 12, wherein the flexible instrument is inserted into the working channel via a fluid-tight valve at a proximal end thereof.

14. The method of claim 12, wherein the device further includes a seal extending around a distal portion of the introducer which, when the device is in the operative position, is seated within and sealingly engages tissue of a cervical os.
